(19) United States
(12) Patent Application Publication
(10) Pub. No.: US 2025/0130150 A1
(43) Pub. Date: Apr. 24, 2025

(54) CONSISTENT CALCIMETRY METHODS AND SYSTEMS UTILIZING COMBINATIONS OF CALCIMETERS AND LABORATORY SHAKERS

(71) Applicant: Schlumberger Technology Corporation, Sugar Land, TX (US)

(72) Inventors: Sangeeth Venugopal, Clamart (FR); Mahdi Ammar, Clamart (FR)

(21) Appl. No.: 18/687,435

(22) PCT Filed: Jan. 27, 2023
(86) PCT No.: PCT/US2023/061433

§ 371 Date: Feb. 28, 2024

Related U.S. Application Data
(60) Provisional application No. 63/304,080, filed on Jan. 28, 2022.

(51) Int. Cl. G01N 7/18; G01N 33/24

(52) U.S. Cl. CPC G01N 7/18; G01N 33/24

(57) ABSTRACT

Systems and methods may determine carbonate content in rock samples and may include shaking a calcimeter having a reaction chamber including at least one pressure sensor and an acid cup with a rock sample therein and recording a first pressure value at or after a first time duration of shaking, wherein the first pressure value is indicative of first gas pressure within the reaction chamber at or after the first time duration of shaking. The systems and methods may also record a second pressure value at or after a second time duration of shaking, wherein the second pressure value is indicative of second gas pressure within the reaction chamber at or after the second duration of shaking, and calculate a carbonate content in the rock sample based on at least one of the recorded first pressure value and the recorded second pressure value.

### CROSS-REFERENCE TO RELATED APPLICATION

This application claims the benefit of and priority to U.S. Patent Application No. 63/304,080, filed on Jan. 28, 2022, which is herein incorporated by reference in its entirety.

### BACKGROUND

It is known in that autocalcimeters are utilized to measure pressure(s) from CO₂ that is generated when samples comprising carbonate rocks are exposed to solutions, blends, or mixtures comprising a known acid, such as, for example, 6M HCl. Traditionally, legacy autocalcimeters utilized in, for example, Geoservices are standalone units which is limited in connecting to data acquisition systems to provide and analyze real time data. Additionally, the legacy autocalcimeters has or provides no provision for a continuous shaking or agitation option to a better mix of the samples which would result in faster and more precise reactions between the known acid and carbonate rocks.

With no shaking or agitating option available to the legacy autocalcimeters, the samples are unable to be agitated throughout the typical 15-minute duration of measurement by the legacy autocalcimeters to ensure that powder of the sample was exposed to the known acid. In view of the disadvantage(s) associated with the legacy autocalcimeters, it is typically expected that conditions in the field will not and/or are not capable of achieving an acceptable degree of accuracy with the type of measurements obtainable with the legacy autocalcimeters. The consistent calcimetry methods and systems disclosed herein may overcome the disadvantage(s) associated with the legacy autocalcimeters by utilizing combinations of calcimeters and laboratory shakers during the pressure measurement durations.

### SUMMARY

This summary is provided to introduce a selection of concepts that are further described below in the detailed description. This summary is not intended to identify key or essential features of the claimed subject matter, nor is it intended to be used as an aid in limiting the scope of the claimed subject matter.

In one or more embodiments, methods may determine carbonate content in rock samples and may comprise externally shaking a calcimeter or performing a shaking process in an integrated part of the calcimeter, wherein the calcimeter has a reaction chamber comprising at least one pressure sensor and an acid cup, wherein the acid cup contains a rock sample and HCl. Further, the methods may comprise recording, with the at least one pressure sensor and an acquisition system in digital communication with the calcimeter, a first pressure value at or after a first time duration of shaking, wherein the first pressure value is indicative of first gas pressure within the reaction chamber at or after the first time duration of shaking, and/or recording, with the at least one pressure sensor and the acquisition system, a second pressure value at or after a second time duration of shaking, wherein the second pressure value is indicative of second gas pressure within the reaction chamber at or after the second duration of shaking.

In some embodiments, the methods may further comprise calculating a carbonate content in the rock sample based on at least one of a calibration model, the recorded first pressure value, and the recorded second pressure value.

In some embodiments, calculating the carbonate content in the rock sample further may comprise calculating at least one of: a calcite content of the rock sample based on the recorded first pressure value; and a dolomite content of the rock sample based on the recorded second pressure value. The term “dolomite” as disclosed herein is defined by its chemical composition which is calcium magnesium carbonate having a CaMg(CO₃)₂ content of greater than about 98 weight percent, wherein weight percent may be referred to hereinafter as “WT (%)” throughout the present disclosure and drawing figures.

In some embodiments, the methods may further comprise calculating both the calcite content of the rock sample based on the recorded first pressure value and the dolomite content of the rock sample based on the recorded second pressure value.

In some embodiments, the second time duration of shaking may be greater than the first time duration of shaking.

In some embodiments, the second time duration of shaking may be at least about 15 minutes.

In some embodiments, the external shaking of the calcimeter or the shaking process in the integrated part of the calcimeter may be continuous and uninterrupted excluding a time period associated with recordation of the first pressure value and/or the second pressure value.

In some embodiments, the methods may further comprise calibrating the calcimeter before the rock sample and the HCl are placed into the acid cup.

In some embodiments, the calibrating the calcimeter may comprise at least one step of the following steps of: preparing sets of combinations of calcite with HCl; placing one set of the prepared sets in the reaction chamber of the calcimeter; shaking the one set in the reaction chamber for a third time duration of shaking that is less than the first time duration of shaking; stopping the shaking after the third time duration of shaking expired and waiting for expiration of the first time duration of shaking; weighing the prepared set after the first time duration of shaking expired; and/or measuring pressure in the reaction chamber after the first time duration of shaking expired. The term “calcite” as disclosed herein is defined by its chemical composition, which is CaCO₃, and the terms “calcite” and “CaCO₃” may be used interchangeably throughout the present disclosure and drawing figures.

In at one embodiment, the calibrating the calcimeter may further comprise sequentially repeating the placing step, the shaking step, the stopping step, the weighing step, and the measuring step for each set of the prepared sets; digitally plotting points indicative of weights of the prepared sets verse measured pressures for the prepared sets; and/or digitally calculating a slope of a best fit line between the digitally plotted points.

In one or more embodiments, methods may determine carbonate content in rock samples and may comprise connecting a calcimeter to an acquisition system or an integrated central processing unit system configured for recording and/or receiving measured pressure values, wherein the calcimeter comprises a reaction chamber and at least one pressure sensor. Further, the methods may comprise placing a rock sample and HCl into an acid cup, placing the acid cup in the reaction chamber of the calcimeter, externally shaking the calcimeter with the acid cup or performing a shaking process in an integrated part of the calcimeter, recording, with the at least one pressure sensor, a first pressure value at or after a first time duration of shaking, wherein the first pressure value is indicative of first gas pressure within the reaction chamber at or after the first time duration of shaking, and/or recording, with the at least one pressure sensor, a second pressure value at or after a second time duration of shaking, wherein the second pressure value is indicative of second gas pressure within the reaction chamber at or after the second duration of shaking.

In some embodiments, the methods may further comprise calculating a carbonate content in the rock sample based on at least one of a calibration model, the recorded first pressure value, and the recorded second pressure value.

In some embodiments, calculating the carbonate content in the rock sample may further comprise calculating at least one of: a calcite content of the rock sample based on the recorded first pressure value; and a dolomite content of the rock sample based on the recorded second pressure value.

In some embodiments, the methods may further comprise calculating both the calcite content of the rock sample based on the recorded first pressure value and the dolomite content of the rock sample based on the recorded second pressure value.

In at least one embodiment, the second time duration of shaking is greater than the first time duration of shaking.

In at least one embodiment, the second time duration is at least about 15 minutes or less.

In at least one embodiment, the external shaking of the calcimeter or the shaking process in the integrated part of the calcimeter is continuous and/or uninterrupted excluding a time period associated with recordation of the first pressure value and/or the second pressure value.

In some embodiments, the methods may further comprise calibrating the calcimeter before placing the rock sample and HCl into the acid cup.

In some embodiments, the calibrating the calcimeter further comprises the steps of: preparing sets of combinations of CaCO3 with HCl; placing one set of the prepared sets in the reaction chamber of the calcimeter; shaking the one set in the reaction chamber for a third time duration of shaking that is less than the first time duration of shaking; stopping the shaking after the third time duration of shaking expired and waiting for expiration of the first time duration of shaking; weighing the prepared set after the first time duration of shaking expired; and/or measuring pressure in the reaction chamber after the first time duration of shaking expired.

In some embodiments, the calibrating the calcimeter further comprises at least one step of the following steps: sequentially repeating the placing step, the shaking step, the stopping step, the weighing step, and the measuring step for each set of the prepared sets; digitally plotting points indicative of weights of the prepared sets verse measured pressures for the prepared sets; and digitally calculating a slope of a best fit line between the digitally plotted points.

### BRIEF DESCRIPTION OF THE DRAWINGS

The present disclosure is best understood from the following detailed description when read with the accompanying Figures. It is emphasized that, in accordance with the standard practice in the industry, various features are not drawn to scale. In fact, the dimensions of the various features may be arbitrarily increased or reduced for clarity of discussion.

FIG. 1 shows a setup combination comprising a calcimeter and a shaker, according to one or more examples of the disclosure.

FIG. 2 is a flowchart illustrating a calcimeter calibration method or process, according to one or more examples of the disclosure.

FIG. 3 is a pressure verses weight graph associated with the calcimeter calibration method or process as shown in FIG. 2, according to one or more examples of the disclosure.

FIG. 4 is a flowchart illustrating a data analysis method or process, according to one or more examples of the disclosure.

FIG. 5 includes formulas utilized for a calculation method or process, according to one or more examples of the disclosure.

FIG. 6 is a table of test results of limestone/sandstone combinations with various weight combinations, according to one or more examples of the disclosure.

FIG. 7 is a reference verses measured calcite graph illustrating the test results for the limestone/sandstone combinations, according to one or more examples of the disclosure.

FIG. 8 is a reference verses measured total carbonates graph illustrating the test results for the limestone/sandstone combinations, according to one or more examples of the disclosure.

FIG. 9 is a table of test results of dolostone/sandstone combinations with various weight combinations, according to one or more examples of the disclosure.

FIG. 10 is a reference/measure dolomite graph illustrating the test results for the dolostone/sandstone combinations, according to one or more examples of the disclosure.

FIG. 11 is a pressure-time graph illustrating test results for two different combinations of limestone and dolostone, according to one or more examples of the disclosure.

### DETAILED DESCRIPTION

Illustrative examples of the subject matter claimed below will now be disclosed. In the interest of clarity, not all features of an actual implementation are described in this specification. It will be appreciated that in the development of any such actual implementation, numerous implementation-specific decisions may be made to achieve the developers' specific goals, such as compliance with system-related and business-related constraints, which will vary from one implementation to another. Moreover, it will be appreciated that such a development effort, even if complex and time-consuming, would be a routine undertaking for those of ordinary skill in the art having the benefit of this disclosure.

Further, as used herein, the article “a” is intended to have its ordinary meaning in the patent arts, namely “one or more.” Also, the phrases “selected from the group consisting of,” “chosen from,” and the like include mixtures of the specified materials. Terms, such as, for example, “contains” and the like are meant to include “including at least” unless otherwise specifically noted.

Herein, the term “about” when applied to a value generally means within the tolerance range of the equipment used to produce the value, or in some examples, means plus or minus 10%, or plus or minus 5%, or plus or minus 3%, or plus or minus 1%, unless otherwise expressly specified. Further, herein the term “substantially” as used herein means a majority, or almost all, or all, or an amount with a range of about 51% to about 100%, for example. Moreover, examples herein are intended to be illustrative only and are presented for discussion purposes and not by way of limitation. Where a numerical limit or range is stated herein, the endpoints are included. Also, all values and subranges within a numerical limit or range are specifically included as if explicitly written out.

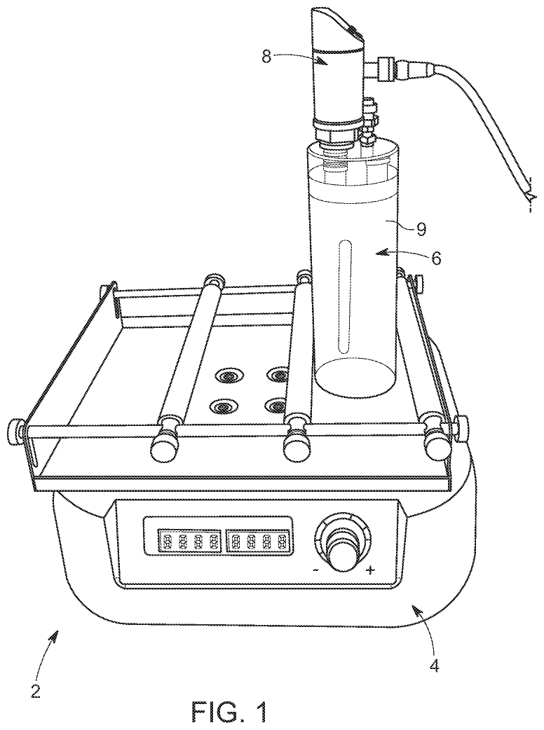

Embodiments disclosed herein relate generally to methods and systems that determine or are configured to determine a carbonate content or, for example, a calcite content and/or a dolomite content (collectively referred to hereinafter as “calcite and dolomite contents”) in at least one rock sample. More specifically, consistent calcimetry methods (hereinafter “methods disclosed herein”) and systems (hereinafter “systems disclosed herein”) utilize at least one combination 2 as shown in FIG. 1. The at least one combination may comprise at least one laboratory shaker 4 and at least one calcimeter 6 to determine the carbonate content and/or the calcite and dolomite contents in one or more rock samples.

In the systems disclosed herein and/or the methods disclosed herein may measure, determine, and/or calculate carbonate contents and/or at least one content of the calcite and dolomite contents in one or more rock samples by utilizing the combination 2 of the at least one calcimeter 6 and the at least one laboratory shaker 4 (collectively referred to hereinafter as “the combination 2 disclosed herein”). The at least one calcimeter 6 measures or is configured to measure pressure(s) from or pressure value(s) associated with CO₂ generated or CO₂ generation when one or more rock samples comprising carbonate-containing rock(s) are exposed to a low or lower concentration of acid or HCl. In some embodiments, the low or lower concentration of HCl may be measured, identified, or associated with a weight % or a concentration % of HCl. The measured pressure(s) and/or pressure value(s) obtained or obtainable by the at least one calcimeter 6 may be utilized to analysis, interpret, calculate, measure, and/or determine mass concentration(s) of carbonate, calcite and/or dolomite in one or more rock samples comprising an unknown amount or concentration of at least one carbonate-containing rock or rock sample. One or more acquisition systems (not shown in the drawings) and/or computing devices or instruments (not shown in the drawings) may be in digital communication with the at least one calcimeter to analyze, interpret, calculate, measure, and/or determine the mass concentration(s) of carbonate, calcite, and/or dolomite in the unknown carbonate-containing rock(s) or rock sample(s) based on the pressure and/or pressure value(s) measured and/or collected by the at least one calcimeter.

In general, known autocalcimeters measure pressure from CO₂ that is or may be generated when at least one carbonate-containing rock or rock sample is exposed to, for example, a concentration of acid. However, the known autocalcimeters utilized in mud logging operations are standalone units which have a limitation with respect to connecting to and/or to communicating with data acquisition systems. In other words, known autocalcimeters are not digitally connected to, digitally connectable to, or in digital communication with data acquisition systems. As a result, the known autocalcimeters utilized during mud logging operations are unable to provide real time data to data acquisition systems with respect to increased pressure(s) from CO₂ that is generated by exposure of the carbonate-containing rock(s) to acid. Also, there is no provision with respect to known autocalcimeters for continuous shaking and/or agitation options or continuous shaking and/or agitation of the known autocalcimeters to improve an acid interaction and/or mixing of the acid with rock sample(s). In contrast, the systems and methods disclosed herein comprise a continuous or substantially continuous shaking or agitation option that results in surprisingly improved acid interactions and/or mixing of the rock sample(s) and acid which may provide or result in faster chemical reactions and/or more reproduceable chemical reactions between the acid and carbonate(s) within the rocks samples.

In contrast to the pressure(s) and/or pressure value(s) measurable by the systems and method disclosed herein that may utilize continuous or substantially continuous shaking and/or agitation options, rock samples measurable by known autocalcimeters are not agitated throughout a full-time duration of measurement(s) to ensure that the rock samples or surfaces of the rock samples are evenly or uniformly exposed to or in contact with the acid. Thus, conditions in the field associated with known autocalcimeters often, if not always, result in failure to achieve a required degree of accuracy for qualitative analytical measurement(s). In some embodiments, the full-time duration of measurement(s) may be or may substantially be at least about 5 minutes, at least about 10 minutes, about 15 minutes, no more than about 20 minutes, or no more than about 25 minutes and/or the required degree of accuracy for qualitative analytical measurement may be or may substantially be greater than about 1% absolute error, greater than about 2% absolute error, about 5% absolute error, less than about 7% absolute error, or less than about 10% absolute error. In at least one embodiment, the concentration of HCl in which the rock sample(s) may be exposed to may be more than about 2M HCl, more than about 4M HCl, about 6M HCl, less than about 8M HCl, or less than about 10M HCl. In one or more embodiments, the concentration of HCl may be greater than about 2% HCl, greater than about 4% HCl, greater than about 6% HCl, greater than about 8% HCl, about 10% HCl, less than about 12% HCl, less than about 14% HCl, less than about 16% HCl, less than about 18% HCl, or less than about 20% HCl.

In one or more embodiments, the systems and methods disclosed herein measure or may measure total carbonates in or contained within one or more rock samples. The one or more rock samples have or may have salt contents that may overwhelmingly exceed a total calcium carbonate content in or within the one or more rock samples. Often, one or more carbonates are present in or contained within one or more various types of oil well cores and/or drilled cuttings. Generally, the oil well cores and/or drilled cuttings may include one or more various types of alkaline earth carbonates and the alkaline earth carbonates that may include, but are not limited to, calcium carbonate (i.e., CaCO₃; also referred to hereafter as “calcite”), magnesium carbonate (i.e., MgCO₃; also referred to hereinafter as “dolomite”), or at least one combination thereof. During the lifecycle of a well, at least one carbonate, such as, calcium carbonate, magnesium carbonate, or a combination thereof, may buildup in one or more drilling fluids and/or one or more water treatment processes (hereinafter “the carbonate buildup”). As a result of the carbonate buildup, one or more scaling problems may develop and/or may be experienced in or exhibited within, for example, at least one tubing, at least equipment component associated with the well, or a combination thereof. Over time, the carbonate buildup may reduce, substantially reduce, and/or cause a complete or substantially complete loss of production in the well comprising the carbonate buildup. Until the scale or carbonate buildup is properly treated, the reduction in, substantial reduction in, or loss of production may continue and/or increase in the well comprising the carbonate buildup. Reduction in, substantial reduction in, or loss of production in the well may ultimately or eventually affect economic viability of the well comprising the carbonate buildup. Therefore, at least one objective of the systems and methods disclosed herein may be to determine a type of carbonate in the carbonate buildup present in the well and/or an amount of carbonate in the carbonate buildup present in the well such that at least one appropriate and/or effective chemical and/or remedial treatment composition may be applied to the well. As a result of application of the chemical and/or remedial treatment composition, the carbonate buildup present in the well may be reduced, substantially reduced, decreased, and/or eliminated within or throughout the well and/or with respect to the at least one tubing, the at least one equipment component associated with the well, or at least one combination thereof.

In one or more embodiments, the present systems and methods may comprise, consist of, include, or incorporate the combination 2 disclosed herein that comprises or consists of the at least one calcimeter 6 (hereinafter “the calcimeter 6”) and the at least one laboratory shaker 4 (hereinafter “the shaker 4”). The systems and methods may achieve one or more objectives with respect to the carbonate buildup by reducing detection times of the total carbonates in the rock samples by an improved factor and/or with a better accuracy when directly compared to standard processes utilizing known autocalcimeters with manual shaking operations. In at least one embodiment, the improved factor achievable or exhibited by the present systems and methods may be or may substantially be a factor of more than about 1.5, more than about 2, about 3, less than about 4, or less than about 5. The present disclosure should not be deemed as limited to a specific embodiment of the improved factor achievable by the systems and methods disclosed herein.

In one or more embodiments, the methods disclosed herein for measuring, determining, analyzing, and/or calculating carbonate contents and/or at least one content of the calcite and dolomite contents in one or more rock samples may be implemented and/or executed with or via one or more systems as disclosed herein (hereinafter “the systems”). In some embodiments, the systems may comprise, consist of, include, and/or incorporate the combination 2 disclosed herein comprising the calcimeter 6 and the shaker 4. As a result of the combination 2 disclosed herein, one or more accurate and/or faster results may be observable or achievable by the systems and methods disclosed herein when directly compared to results observed or achieved by known and/or conventional calcimetry systems and/or measuring methods. The one or more systems and methods disclosed herein may further comprise the acquisition system that may be connected or connectible to the combination 2, the calcimeter 6, and/or the shaker 4. In some embodiments, the acquisition system may be in digital communication with the calcimeter 6 such that digital output from the calcimeter 6 may be outputted or transmitted directly or indirectly to the acquisition system. In at least one embodiment, the calcimeter 6 may be directly and/or digitally connected to the acquisition system such that continuous data may be recorded for any/all analysis, tests, and/or experiments conducted and/or completed by the systems and methods disclosed herein. As a result of the recordation of the continuous data, each analysis of continuous data may be digitally plotted or illustrated as one or more digital graphs, tables, and/or charts. In an embodiment, the digital plotted or illustrated graphs may be one or more pressure verses time graphs.

The calcimeter 6 disclosed herein may comprise at least one pressure sensor 8, a reaction chamber 10, at least one valve in fluid communication with the reaction chamber, or at least one combination thereof. In some embodiments, the at least one pressure sensor 8 may be in communication with the reaction chamber 10, at least partially disposed within the reaction chamber 10, and/or configured or adapted to measure or obtain pressure(s) and/or pressure value(s) present within the reaction chamber 10 of the calcimeter 6. The reaction chamber 10 may be sized, shaped, configured, and/or adapted to receive an acid cup that may contain at least one carbonate-containing rock sample comprising a carbonate content or calcite and/or dolomite contents. In an embodiment, the at least one pressure sensor 8 may be a digital pressure sensor and the at least one valve may be a relief valve for the reaction chamber 10. In some embodiments, the shaker 4 may comprise a platform that is or may be sized, shaped, configured, and/or adapted to receive the calcimeter 6 thereon. After the calcimeter 6 is received onto the platform of the shaker 4, the shaker 4 may shake and/or agitate the calcimeter 6, the acid cup received in the reaction chamber 10, the rock sample contained within the acid cup, or at least one combination thereof to calculate, determine, analyze, and/or measure the carbonate content or the calcite and/or dolomite contents of the rock sample. In an embodiment, pressure(s) recordable via the at least one pressure sensor 10 after the shaker 4 agitates and/or shakes the calcimeter 6 and/or rock sample may be utilized to calculate, determine, analyze, and/or measure the carbonate content in the rock sample, carbonates in the rock sample, and/or calcite and dolomite contents in the rock sample, In at least one embodiment, the shaker 4 may be an orbital shaker that may comprise electronic digital controls of at least one of speed, run-time, rotation frequency, rotation amplitude, or a combination thereof.

In one or more embodiments, at least one rock sample to be measured and/or analyzed may be crushed and reacted with the concentration of acid to form CO₂. A build-up of pressure from the formation of CO₂ (hereinafter “the pressure buildup”) may be measured and/or compared against a pressure build-up from a calibration sample, a set comprising a plurality of calibration sample, and/or at least one calibration set comprising pure or substantially pure calcium carbonate that has undergone the same or substantially the same reaction with the acid. In some embodiments, the acid may be HCl, such as, for example, 10% HCl. The percentage of calcium in the rock sample is a function of the measured pressure build-up such that the percentage of calcium may be a relative measurement based on the measured pressure build-up. Moreover, the carbonate content in the rock sample, the carbonates in the rock sample, and/or the calcite and dolomite contents in the rock sample may be calculated, determined, analyzed, and/or measured based upon the percentage of calcium in the rock sample, the measured pressure build-up, or a combination thereof. In at least one embodiment, the systems and methods disclosed herein may utilize and/or execute computer modeling of calcimeter pressure build-ups to improve calcite estimations as disclosed in U.S. Pat. No. 9,234,827 B2, which is incorporated by reference herein in its entirety.

In one or more embodiments, the systems and methods disclosed herein comprising the combination 2 provides, achieves, and exhibits, a plurality of surprising and unexpected advantages and/or improvements over known analysis systems and methods. The plurality of advantages and/or improvements may comprise improved time reductions for analysis of carbonate contents of rock samples, improved real-time data recordings of the measured pressures or pressure values indicative of the generated CO₂, unexpected process simplifications, improved measurement consistencies, improved health, safety, and environment (hereinafter “HSE”) risk reductions, or at least one combination thereof. With respect to the improved time reductions for analysis, the systems and methods disclosed herein may save valuable time via, for example, a fifteen (15) minute analysis time which may be necessary and/or required to complete or execute a carbonate content analysis of rock samples. As a result, the analysis of rock samples on drilling rigs may be more timely, efficient, and/or cost-effective. The improved real-time data recordings may facilitate pressure readings that may be automatically saved in the acquisition system to calculate concentrations of calcite and dolomite within rock samples. The unexpected process simplifications of the methods disclosed herein may utilize the shaker 4 which may avoid intermediate manual shakings by operators thereby removing human errors and avoiding unnecessary discrepancies cause by or due to irregular shaking patterns. The improved measurement consistencies may be achievable by the systems and methods disclosed herein based on constant, consistent, and/or reliable shaking patterns performed or executed by the shaker 4. The improved HSE risk reductions may be achievable by the systems and methods disclosed herein because the low or lower concentrations of HCl that are utilized during the present methods and that the acid cups, after the initial mixing of the rock samples and acids, may be placed on the shaker 4 to avoid any human contacts until the chemical reactions between the rock samples and the acids are finished, completed, or terminated. In an embodiment, the present methods may utilize, for example, 10% HCl which is lower or substantially lower than 50% HCl which is utilized by known analysis methods comprising known autocalcimeters.

The improved and unexpected advantages achievable by the systems and methods disclosed herein are in no manner limited to the above-mentioned advantages.

### EXAMPLES

The following examples, tests, and/or experiments are illustrative of and do not limit the systems and methods disclosed herein. In the examples disclosed herein after, the combination of the systems and methods comprises a calcimeter and a laboratory shaker having parameters as set forth hereafter. The pressure sensor(s) of the calcimeter has an output that ranges from about 4 mA to about 20 mA or about 0 V to about 10 V, the calcimeter comprise a pressure sensor configured for about 2.5 Bar and an acid cup of about 20 ml, and the outputs of the calcimeter comprises calcite concentration, dolomite concentration, or the combination thereof. The laboratory shaker has a rotation frequency ranging from about 50 rpm to about 250 rpm, a rotational amplitude of about 20 mm, a load capacity of up to about 25 kg, an operational timer ranging from about 1 minutes up to about 99 hours and 59 minutes, and dimensions of about 10 cm high, about 28 cm wide, and about 26 cm deep.

The system disclosed herein for the examples was assembled or setup via an initial setup method. The initial setup method for the system disclosed herein comprises the following steps: connecting the pressure sensor of the calcimeter to an acquisition system via a 4-20 mA channel; powering up or activating the calcimeter via a 24 V power supply; and powering up or activating a weighing balance and the laboratory shaker.

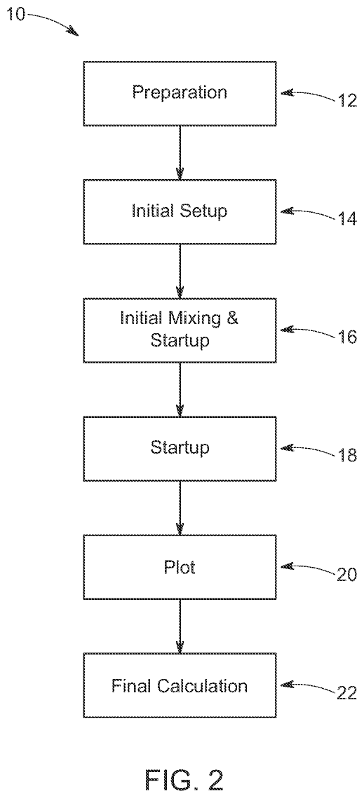

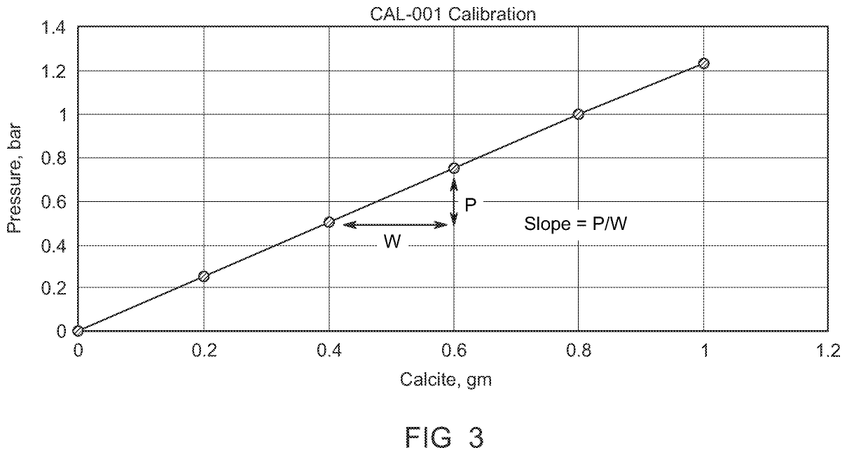

FIG. 2 illustrates a calibration method 10 for calibrating the calcimeter, wherein the calibration method 10 comprises one or more of the following steps: a preparation step 12; an initial setup step 14; an initial mixing & startup step 16; a startup step 18; a plot step 20; and/or a calculation step 22. For the calibration method 10, a plurality of sets of various combinations of pure CaCO₃ with a lower concentration of HCl were prepared and, upon completion of the chemical reaction, a slope was calculated utilizing a pressure verses weight graph as shown in FIG. 3. In the preparation step 12, a set of four combinations having different pure CaCO₃ concentrations (i.e., 0.2 gm, 0.4 gm, 0.6 gm, and 0.8 gm of pure CaCO₃ powder) mixed with 10% HCl were prepared in the acid cup for analysis. With respect to the initial setup step 14, the reaction chamber comprising 0.2 gm of CaCO₃ powder along with 20 ml HCl was placed or disposed within the reaction chamber. The acid cup placed inside the reaction chamber was shaken or agitated by the laboratory shaker for 10 seconds to produce or have a complete mixture of the sample during the initial mixing at startup step 16. After waiting 30 seconds at the end of step 16 and/or the initial mixing, the pressure buildup within the reaction chamber was measured and/or recorded for each reaction at the startup step 18. The startup step 18 was repeated for each of the other three combinations. A calibration curve was plotted during the plot step 20 via the pressure verse weight graph shown in FIG. 3, wherein the slope M of the calibration curve is Y/X, Y is a pressure reading difference between two points, and X is a difference in grams between two points, wherein the unit “gram” or “grams” may be referred to hereinafter as “gm” throughout the present disclosure and drawing figures. In the calculation step 22, an average of the four slope readings is calculated to determine or measure a final slope of the calibration curve as shown in FIG. 3.

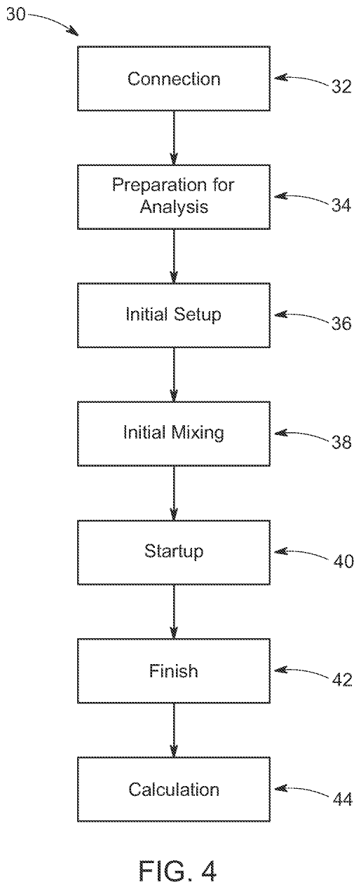

FIG. 4 illustrates a procedural analysis method 30 for analyzing the carbonate content or calcite and/or dolomite contents in rock samples analyzed by the systems and methods disclosed herein. The procedural analysis method 30 comprises one or more of the following steps: a connection step 32; a preparation for analysis step 34; an initial setup step 36; an initial mixing step 38; a startup step 40; a finish step 42; and/or a calculation step 44. In the examples disclosed herein, the combination of the calcimeter and the laboratory shaker was utilized for the analysis set forth in the procedural analysis method 30. The procedural analysis method 30 improved analysis time by about thirty minutes when compared to known analysis methods utilizing known autocalcimeters and obtained better and/or higher quality results when compared to known analysis methods utilizing known autocalcimeters.

During the connection step 32, a calcimeter was powered up and connected to an acquisition system in addition to the laboratory shaker and weighing balance being powered up. About 1 gram of rock sample was prepared and mixed with 10% HCl in the acid cup during the preparation at analysis step 34 and the acid cup was subsequently placed inside the reaction chamber before the reaction chamber was closed at the initial setup step 36. In the initial mixing step 38, the calcimeter comprising the acid cup was shaken or agitated for ten (10) seconds to form or produce a complete mix of the rock sample and HCl acid. The laboratory shaker was started and the calcimeter was placed or put on the laboratory shaker, wherein the laboratory shaker agitates or shakes the calcimeter with a constant speed of 105 rpm at the startup step 40. During the finish step 42, the laboratory shaker continued to shake or agitate the calcimeter for fifteen (15) minutes, wherein pressures or pressure values were automatically measured and recorded at thirty (30) seconds of shaking and fifteen (15) minutes of shaking via the pressure sensor of the calcimeter and the acquisition system. A calcite content of the rock sample was calculated and/or determined based on the pressure or pressure value measured and/or recorded at the thirty (30) second reading and a dolomite content of the rock sample was calculated and/or determined based on the pressure or pressure value measured and/or recorded at the fifteen (15) minute reading at the calculation step 44.

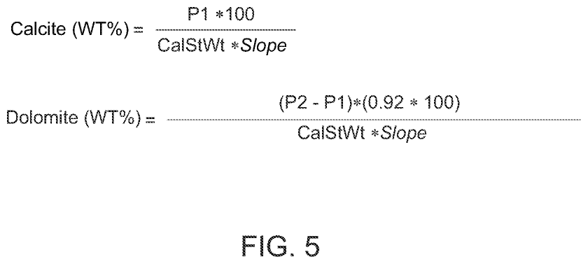

FIG. 5 sets for an equation for calculating the calcite content (i.e., weight % of calcite or calcite (wt. %)) in the rock sample and the dolomite content (i.e., weight % of dolomite (wt. %)) in the rock sample. In the equations set forth in FIG. 5, P1 represents total pressure measured in bar at 30 seconds from reaction initiation, slope is in bar/g, P2 represents total pressure measured in bar at 15 minutes from reaction initiation, and CalStWt refers to 1 gram of the rock sample.

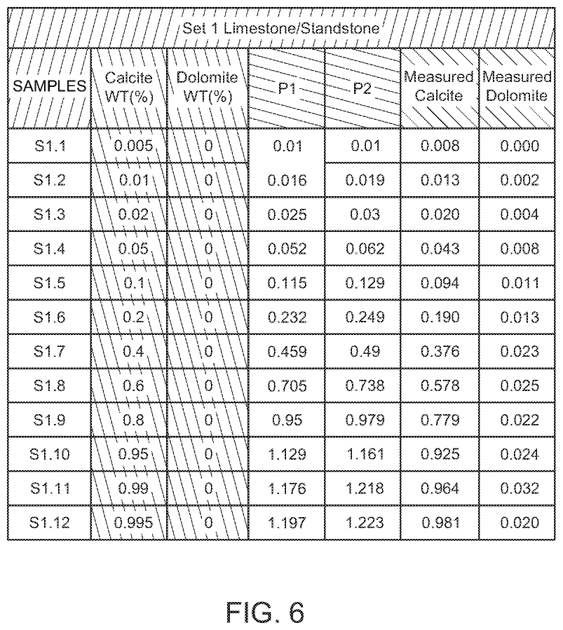

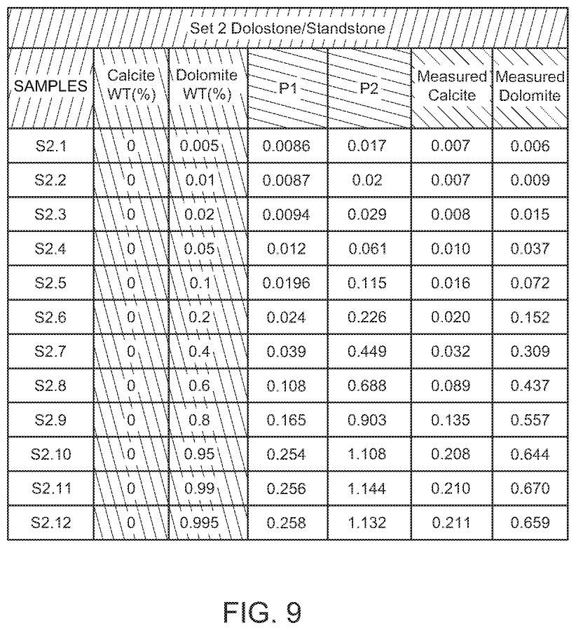

For the examples disclosed herein, a series of tests/experiments with predefined combinations of samples (i.e., Sample Set 1/Sample Set 2 as shown in FIG. 6 and Sample 2 as shown in FIG. 9) were analyzed and the results were both surprising and unexpected when directly compared to results that may be achievable via known and/or conventional analysis methods. The tests/experiments were executed and/or completed with the following combinations of samples: limestone/sandstone; dolostone/sandstone; limestone/siliceous mudrock; limestone/dolostone; sandstone/calcareous mudrock; and pure dolomite/calcite.

The experimental results for tests/experiments of limestone/sandstone with a plurality of weight combinations are set forth in FIG. 6.

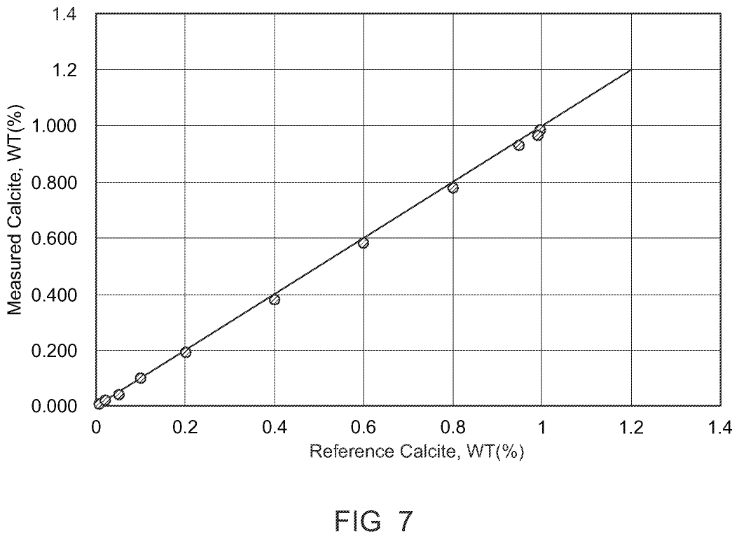

FIG. 7 graphically shows a comparison of reference calcite WT (%) and measured calcite content of the rock sample. As shown in the graph, the test/experimental results have or show an unexpectedly good and improved correlation and an improved low error when directly compared to the reference data shown therein.

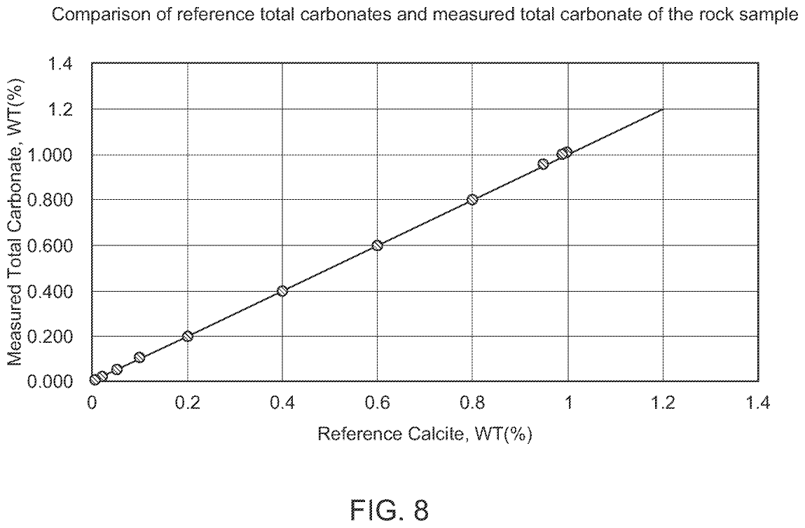

FIG. 8 graphically shows a comparison of reference total carbonates WT (%) and measured total carbonate WT (%) of the rock sample. As shown in the graph, the test/experimental results have or show an unexpectedly good and improved correlation and an improved low error when directly compared to the reference data shown therein.

The experimental results for tests/experiments of dolostone/sandstone with a plurality of weight combinations are set forth in FIG. 9.

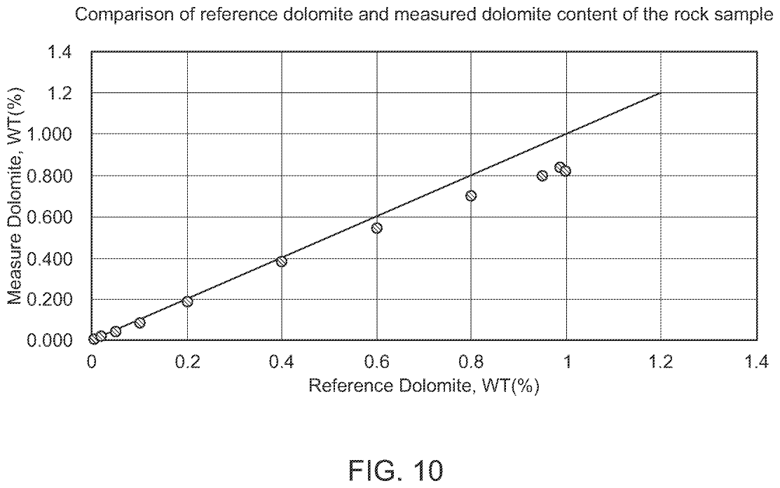

FIG. 10 graphically shows a comparison of reference dolomite WT (%) and measured dolomite content of the rock sample. As shown in the graph, the test/experimental results have or show an unexpectedly good and improved correlations when directly compared to a conventional calcimeter and an improved low error when directly compared to the reference data shown therein. Moreover, in some embodiments, the accuracy of the calibration model disclosed herein may be further improved by using or utilizing the calibration method as discussed and described in U.S. Pat. No. 9,234,827 B2.

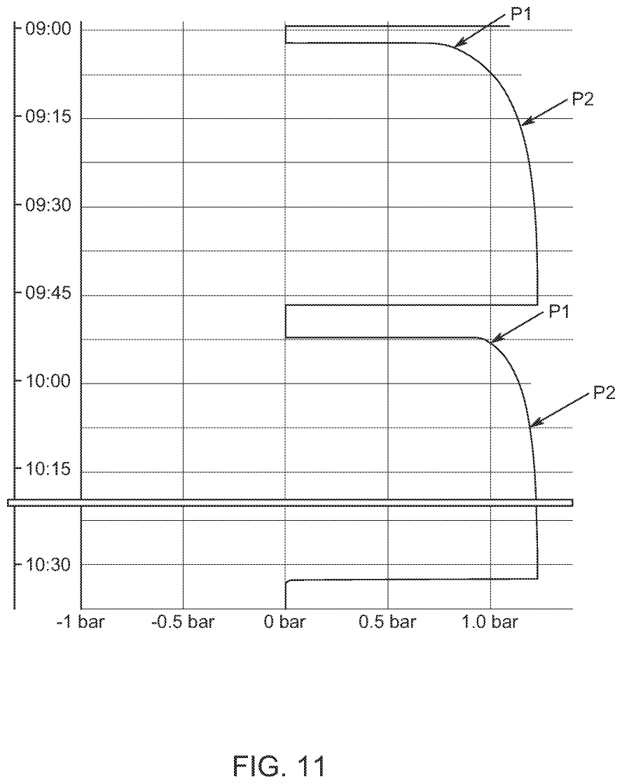

FIG. 11 is a graph illustrating corresponding pressures for P1 and P2 for a first sample combination of limestone and dolostone and a second sample combination of limestone and dolostone. The first sample combination comprised 0.6 gm of limestone and 0.4 gm of dolostone, wherein P1 was 0.783 bar and P2 was 1.152 bar. The second sample combination comprised 0.8 gm of limestone and 0.2 gm of dolostone, wherein P1 was 0.975 bar and P2 was 1.19 bar.

The foregoing description, for purposes of explanation, used specific nomenclature to provide a thorough understanding of the disclosure. However, it will be apparent to one skilled in the art that the specific details are not required in order to practice the systems and methods described herein. The foregoing descriptions of specific examples are presented for purposes of illustration and description. They are not intended to be exhaustive of or to limit this disclosure to the precise forms described. Obviously, many modifications and variations are possible in view of the above teachings. The examples are shown and described in order to best explain the principles of this disclosure and practical applications, to thereby enable others skilled in the art to best utilize this disclosure and various examples with various modifications as are suited to the particular use contemplated. It is intended that the scope of this disclosure be defined by the claims and their equivalents below.

What is claimed is:

1. A method of determining carbonate content in rock samples comprising:
externally shaking a calcimeter or performing a shaking process in an integrated part of the calcimeter, wherein the calcimeter has a reaction chamber comprising at least one pressure sensor and an acid cup, wherein the acid cup contains a rock sample and HCl;
recording, with the at least one pressure sensor and an acquisition system in digital communication with the calcimeter, a first pressure value at or after a first time duration of shaking, wherein the first pressure value is indicative of first gas pressure within the reaction chamber at or after the first time duration of shaking; and
recording, with the at least one pressure sensor and the acquisition system, a second pressure value at or after a second time duration of shaking, wherein the second pressure value is indicative of second gas pressure within the reaction chamber at or after the second duration of shaking.

2. The method of claim 1, further comprising:
calculating a carbonate content in the rock sample based on at least one of a calibration model, the recorded first pressure value, and the recorded second pressure value.

3. The method of claim 2, wherein calculating the carbonate content in the rock sample further comprises:
calculating at least one of:
a calcite content of the rock sample based on the recorded first pressure value; and
a dolomite content of the rock sample based on the recorded second pressure value.

4. The method of claim 3, further comprising:
calculating both the calcite content of the rock sample based on the recorded first pressure value and the dolomite content of the rock sample based on the recorded second pressure value.

5. The method of claim 1, wherein the second time duration of shaking is greater than the first time duration of shaking.

6. The method of claim 1, wherein the second time duration of shaking is at least about 15 minutes.

7. The method of claim 1, wherein the external shaking of the calcimeter or the shaking process in the integrated part of the calcimeter is continuous and uninterrupted excluding a time period associated with recordation of the first pressure value and/or the second pressure value.

8. The method of claim 1, further comprising:
calibrating the calcimeter before the rock sample and the HCl are placed into the acid cup.

9. The method of claim 8, wherein the calibrating the calcimeter further comprises the steps of:
preparing sets of combinations of CaCO3 with HCl;
placing one set of the prepared sets in the reaction chamber of the calcimeter;
shaking the one set in the reaction chamber for a third time duration of shaking that is less than the first time duration of shaking;
stopping the shaking after the third time duration of shaking expired and waiting for expiration of the first time duration of shaking;
weighing the prepared set after the first time duration of shaking expired; and
measuring pressure in the reaction chamber after the first time duration of shaking expired.

10. The method of claim 9, wherein the calibrating the calcimeter further comprises:
sequentially repeating the placing step, the shaking step, the stopping step, the weighing step, and the measuring step for each set of the prepared sets;
digitally plotting points indicative of weights of the prepared sets verse measured pressures for the prepared sets; and
digitally calculating a slope of a best fit line between the digitally plotted points.

11. A method of determining carbonate content in rock samples, comprising:
connecting a calcimeter to an acquisition system or an integrated central processing unit system configured for recording and/or receiving measured pressure values, wherein the calcimeter comprises a reaction chamber and at least one pressure sensor;
placing a rock sample and HCl into an acid cup;
placing the acid cup in the reaction chamber of the calcimeter;
externally shaking the calcimeter with the acid cup or performing a shaking process in an integrated part of the calcimeter;
recording, with the at least one pressure sensor, a first pressure value at or after a first time duration of shaking, wherein the first pressure value is indicative of first gas pressure within the reaction chamber at or after the first time duration of shaking; and
recording, with the at least one pressure sensor, a second pressure value at or after a second time duration of shaking, wherein the second pressure value is indicative of second gas pressure within the reaction chamber at or after the second duration of shaking.

12. The method of claim 11, further comprising:
calculating a carbonate content in the rock sample based on at least one of a calibration model, the recorded first pressure value, and the recorded second pressure value.

13. The method of claim 12, wherein calculating the carbonate content in the rock sample further comprises:
calculating at least one of:
a calcite content of the rock sample based on the recorded first pressure value; and
a dolomite content of the rock sample based on the recorded second pressure value.

14. The method of claim 13, further comprising:
calculating both the calcite content of the rock sample based on the recorded first pressure value and the dolomite content of the rock sample based on the recorded second pressure value.

15. The method of claim 11, wherein the second time duration of shaking is greater than the first time duration of shaking.

16. The method of claim 11, wherein the second time duration is at least about 15 minutes.

17. The method of claim 11, wherein the external shaking of the calcimeter or the shaking process in the integrated part of the calcimeter is continuous and/or uninterrupted excluding a time period associated with recordation of the first pressure value and/or the second pressure value.

18. The method of claim 11, further comprises:
calibrating the calcimeter before placing the rock sample and HCl into the acid cup.

19. The method of claim 18, wherein the calibrating the calcimeter further comprises the steps of:
preparing sets of combinations of CaCO3 with HCl;
placing one set of the prepared sets in the reaction chamber of the calcimeter;
shaking the one set in the reaction chamber for a third time duration of shaking that is less than the first time duration of shaking;
stopping the shaking after the third time duration of shaking expired and waiting for expiration of the first time duration of shaking;
weighing the prepared set after the first time duration of shaking expired; and
measuring pressure in the reaction chamber after the first time duration of shaking expired.

20. The method of claim 19, wherein the calibrating the calcimeter further comprises:
sequentially repeating the placing step, the shaking step, the stopping step, the weighing step, and the measuring step for each set of the prepared sets;
digitally plotting points indicative of weights of the prepared sets verse measured pressures for the prepared sets; and
digitally calculating a slope of a best fit line between the digitally plotted points.
